(19) United States
(12) Patent Application Publication
(10) Pub. No.: US 2023/0100844 A1
(43) Pub. Date: Mar. 30, 2023

(54) IBOGAINE COMBINATION TREATMENT

(71) Applicant: ATAI Life Sciences AG, Berlin (DE)

(72) Inventors: Srinivas G. RAO, Encinitas, CA (US); Glenn SHORT, Scituate, MA (US); Carrie BOWEN, Uxbridge, MA (US); Robert BUSBY, Hingham, MA (US)

(21) Appl. No.: 17/941,648

(22) Filed: Sep. 9, 2022

Related U.S. Application Data
(60) Provisional application No. 63/242,926, filed on Sep. 10, 2021.

(51) Int. Cl. A61K 31/55; A61K 31/137; A61K 31/138; A61K 31/4709; A61K 31/357; A61K 31/4525; A61K 31/4164; A61K 31/454; A61K 31/166; A61K 31/343; A61P 25/30; A61K 31/58; A61K 31/635; A61K 31/4706; A61K 31/5415; A61K 31/5513; A61K 31/704; A61K 31/426; A61K 31/5377; A61K 31/502; A61K 31/506

(52) U.S. Cl. CPC A61K 31/55; A61K 31/137; A61K 31/138; A61K 31/4709; A61K 31/357; A61K 31/4525; A61K 31/4164; A61K 31/454; A61K 31/166; A61K 31/343; A61P 25/30; A61K 31/58; A61K 31/635; A61K 31/4706; A61K 31/5415; A61K 31/5513; A61K 31/704; A61K 31/426; A61K 31/5377; A61K 31/502; A61K 31/506

(57) ABSTRACT

The present disclosure is directed to methods of improving the therapeutic effectiveness and safety profile of ibogaine for the treatment of conditions including, but not limited to, alcoholism, substance abuse disorder, and opioid use disorder.

### CROSS-REFERENCE TO RELATED APPLICATION

The present application claims the benefit of priority to U.S. Provisional Application No. 63/242,926, filed Sep. 10, 2021, the contents of which are hereby incorporated by reference in its entirety.

### BACKGROUND OF THE INVENTION

Ibogaine is a naturally-occurring psychoactive compound found in the root bark of the African shrub Tabernanthe iboga, which has been used for hundreds to thousands of years in ceremonial and spiritual settings in Africa. Ibogaine can produce dream-like visualizations and perceptual changes that subside within 6-8 h, consistent with the time course of clearance of ibogaine from the blood (Mash et al., 2018). Studies in humans and animals indicate the potential utility of ibogaine for the treatment of substance use disorders. In particular, a meta-analysis of animal research showed that ibogaine reduced self-administration of several drugs, including opiates, cocaine, and ethanol (Belgers et al., 2016). In an open-label study in opioid- and cocaine-dependent subjects, single oral administration of ibogaine (8-12 mg/kg HCl) reduced opioid withdrawal signs and craving and increased mood (Mash et al., 2018). The pharmacological basis for these effects is unclear, as ibogaine exhibits complex interactions with serotonin, dopamine, acetylcholine, opioid, sigma, glutamate and other receptor systems in the central nervous system (Wasko et al., 2018).

Despite these benefits, the pharmacokinetic profile of ibogaine potentially limits its therapeutic utility. Following oral administration, ibogaine is rapidly metabolized by CYP2D6 in the gut wall and liver (Koenig and Hilber, 2015) to its primary metabolite, noribogaine. Noribogaine is non-hallucinogenic compound, which has an overlapping, but distinct profile of pharmacological effects. Oral administration of ibogaine has been associated with cardiac QT interval prolongation, which may enhance the risk for arrhythmia. As this cardiovascular event has been observed days after ibogaine has been cleared from the body, it is believed that these adverse effects are primarily mediated by noribogaine, which is known to exhibit a longer half-life (Henstra et al., 2017; Glue et al., 2015).

Accordingly, in order to harness ibogaine's full therapeutic potential, methods are needed to increase and prolong exposure to ibogaine, while reducing exposure to noribogaine.

### BRIEF SUMMARY OF THE INVENTION

In one aspect, the present disclosure provides a method of increasing the bioavailability of ibogaine in a patient in need thereof, the method comprising administering to the patient: (a) a drug that inhibits the metabolism of ibogaine and (b) a therapeutically effective amount of ibogaine, or a pharmaceutically acceptable salt thereof.

In one aspect, the present disclosure provides a method of treating a condition that is treatable with ibogaine in a patient in need thereof, the method comprising administering to the patient: (a) a drug that inhibits the metabolism of ibogaine and (b) a therapeutically effective amount of ibogaine, or a pharmaceutically acceptable salt thereof.

In some embodiments, the condition that is treatable with ibogaine is selected from the group consisting of alcoholism, substance abuse disorder, and opioid use disorder. In some embodiments, the condition is opioid use disorder.

In some embodiments, the drug that inhibits the metabolism of ibogaine is a CYP2D6 inhibitor. In some embodiments, the CYP2D6 inhibitor is selected from the group consisting of abiraterone, amiodarone, bupropion, celecoxib, chloroquine, chlorpromazine, cimetidine, cinacalcet, citalopram, clobazam, clozapine, cobicistat, desvenlafaxine, diltiazem, diphenhydramine, doxorubicin, duloxetine. Echinacea, escitalopram, febuxostat, fluoxetine, fluphenazine, Gingko biloba, fluvoxamine, gefitinib, haloperidol, hydralazine, hydroxychloroquine, imatinib, labetalol, lansoprazole, lorcaserin, metoclopramide, methadone, mirabegron, olanzapine, Panax ginseng, paroxetine, pazopanib, perhexiline, propafenone, progesterone, propoxyphene, quinidine, ranitidine, risperidone, ritonavir, sertraline, telithromycin, terbinafine, terfenadine, testosterone, thioridazine, trifluperidol, verapamil, vemurafenib. In some embodiments, the CYP2D6 inhibitor is bupropion or escitalopram and enantiomers thereof. In some embodiments, the CYP2D6 inhibitor is fluoxetine. In some embodiments, the CYP2D6 inhibitor is quinidine.

In some embodiments, the CYP2D6 inhibitor is administered within about 12 h of administration of the ibogaine or a pharmaceutically acceptable salt thereof. In some embodiments, the CYP2D6 inhibitor is administered 5 to 7 days prior to administration of the ibogaine or a pharmaceutically acceptable salt thereof. In some embodiments, the CYP2D6 inhibitor is co-administered with the ibogaine or a pharmaceutically acceptable salt thereof.

In some embodiments, the drug that inhibits the metabolism of ibogaine is a CYP2D6 inactivator. In some embodiments, the CYP2D6 inactivator is selected from the group consisting of 3,4-Methylenedioxymethamphetamine (MDMA), paroxetine, cimetidine, pimozide, methamphetamine, metoclopramide or desethylamiodarone.

In some embodiments, the CYP2D6 inactivator is co-administered with the ibogaine or a pharmaceutically acceptable salt thereof. In some embodiments, the CYP2D6 inactivator is administered at least 1 day prior to ibogaine or a pharmaceutically acceptable salt thereof. In some embodiments, the patient is pre-treated with the drug that inhibits ibogaine metabolism prior to administration of the ibogaine. In some embodiments, the patient is pre-treated with the drug that inhibits ibogaine metabolism at least 3 days prior (e.g., about 5 days prior or 7 days prior) to administration of ibogaine.

In some embodiments, the administration of the drug that inhibits the metabolism of ibogaine reduces the patient's systemic exposure to noribogaine compared to a patient administered the same dose of ibogaine without administration of the drug that inhibits the metabolism of ibogaine. In some embodiments, the administration of the drug that inhibits the metabolism of ibogaine increases the patient's systemic exposure to ibogaine compared to a patient administered the same dose of ibogaine without administration of the drug that inhibits the metabolism of ibogaine.

In some embodiments, the administration of the drug that inhibits the metabolism of ibogaine reduces the patient's Cmax of noribogaine compared to a patient administered the same dose of ibogaine without administration of the drug that inhibits the metabolism of ibogaine. In some embodiments, the administration of the drug that inhibits the metabolism of ibogaine increases the patient's Cmax of ibogaine compared to a patient administered the same dose of ibogaine without administration of the drug that inhibits the metabolism of ibogaine.

### Definitions

While the following terms are believed to be well understood by one of ordinary skill in the art, the following definitions are set forth to facilitate explanation of the presently disclosed subject matter.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

“Ibogaine” refers to a compound having the structural formula:

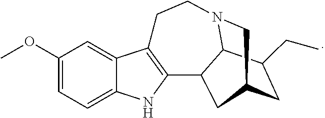

Ibogaine is isolated from Tabernanth iboga, a shrub of West Africa. Ibogaine can also be synthesized using known methods. See, e.g., Büchi, et al. (1966), J. Am. Chem Society, 88(13), 3099-3109.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” as used herein with regard to a patient, refers to improving at least one symptom of the patient's disorder. Treating can be curing, improving, or at least partially ameliorating a disorder. For purposes of the present disclosure, treating includes, but is not limited to curing, improving, or at least partially ameliorating alcoholism, substance abuse disorder, and opioid use disorder.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

“Therapeutically effective amount”, “effective amount” or “therapeutic amount” refers to an amount of drug(s) or agent(s) that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations.

As used herein, the term “patient” or “subject” refers to mammals and includes humans and non-human mammals.

As used herein, “CYP2D6 inhibitor” refers to a compound that reversibly binds to CYP2D6 and inhibits the metabolic activity of this enzyme towards endogenous and xenobiotic compounds. Non-limiting examples of CYP2D6 inhibitors include abiraterone, amiodarone, bupropion, celecoxib, chloroquine, chlorpromazine, cimetidine, cinacalcet, citalopram, clobazam, clozapine, cobicistat, desvenlafaxine, diltiazem, diphenhydramine, doxorubicin, duloxetine, Echinacea, escitalopram, febuxostat, fluoxetine, fluphenazine, Gingko biloba, fluvoxamine, gefitinib, haloperidol, hydralazine, hydroxychloroquine, imatinib, labetalol, lansoprazole, lorcaserin, metoclopramide, methadone, mirabegron, olanzapine, Panax ginseng, paroxetine, pazopanib, perhexiline, propafenone, progesterone, propoxyphene, quinidine, ranitidine, risperidone, ritonavir, sertraline, telithromycin, terbinafine, terfenadine, testosterone, thioridazine, trifluperidol, verapamil, vemurafenib.

As used herein, “CYP2D6 inactivator” is a compound that inhibits the activity of the CYP2D6 in a mechanism-based (irreversible) manner. In some embodiments, a compound of this type undergoes metabolic bioactivation by CYP2D6 to an electrophilic intermediate, which causes quasi-irreversible (e.g., by forming metabolic-intermediate complex (MIC)) or irreversible (e.g., by formation of a covalent bond between metabolite and enzyme) inhibition of the enzyme. Non-limiting examples of CYP2D6 inactivators include MDMA, paroxetine, cimetidine, pimozide, methamphetamine, metoclopramide or desethylamiodarone.

As used herein, the term “QT interval” refers to the measure of the time between the start of the Q wave and the end of the T wave in the electrical cycle of the heart. Prolongation of the QT interval refers to an increase in the QT interval.

### DETAILED DESCRIPTION OF PREFERRED EMBODIMENTS

Ibogaine is a naturally-occurring psychoactive compound that has potential utility in the treatment of substance use disorders and related conditions. Following single oral administration of ibogaine to humans, blood concentrations of ibogaine peaked within a couple of hours and were detectable for about 24 h. However, ibogaine is rapidly metabolized by CYP2D6 in the gut wall and liver to noribogaine, an O-demethylated non-hallucinogenic compound with an overlapping, but distinct pharmacological profile compared to the parent compound. Concentrations of noribogaine peaked later and were detectable for a longer duration (Mash et al., 2011; Glue et al., 2015).

CYP2D6 is known to have phenotypic variability, which means that subjects can exhibit levels of metabolic activity that depend on their enzyme function. In some embodiments, subjects are characterized as poor metabolizers (i.e., have little or no CYP2D6 function), intermediate metabolizers (i.e., metabolize drugs at a rate somewhere between the poor and extensive metabolizers), extensive metabolizers (i.e., normal CYP2D6 function), or ultrarapid metabolizers (i.e., greater than normal CYP2D6 function, for example from multiple copies of the CYP2D6 gene).

In a study by Mash et al. (2011), human drug-dependent patient volunteers who were classified as CYP2D6 extensive metabolizers and received a single oral dose of ibogaine (10 mg/kg) exhibited CYP2D6-mediated metabolism of ibogaine that resulted in high levels of noribogaine in blood, with Cmax values in the same range as the parent drug. The majority (>90%) of absorbed ibogaine was eliminated by 24 hours post dose, while the concentrations of noribogaine remained elevated at 24 hours. In CYP2D6 poor metabolizers that received a single oral dose of ibogaine (10 mg/kg), exposure to ibogaine was modestly increased and exposure to noribogaine was markedly decreased compared to extensive metabolizers. In the study by Glue et al. (2015), healthy male volunteers characterized as CYP2D6 extensive metabolizers who were chronically treated with the CYP2D6 inactivator paroxetine (10-20 mg) and received a single ibogaine administration (low dose of 20 mg or about 0.3 mg/kg) exhibited increased plasma exposures to ibogaine and decreased plasma exposures to noribogaine, with detectable ibogaine plasma exposures out to 72 h (vs. <4 h with chronic placebo treatment).

Accordingly, in some embodiments, the effect of administering the combination of CYP2D6 inhibitor or inactivator with ibogaine, as described herein, is to normalize exposures to ibogaine and noribogaine across subjects/patients of all CYP2D6 phenotypes to achieve a more homogenous therapeutic response.

To achieve this goal and enhance the therapeutic effectiveness of ibogaine, the present disclosure describes methods of increasing and prolonging exposure to ibogaine, while reducing exposure to noribogaine—a primary metabolite suspected to play a role in QT prolongation that may increase the risk for cardiac arrhythmia. By improving the pharmacokinetic profile in this manner, the methods disclosed herein allow a lower therapeutically effective dose of ibogaine to be administered to a patient in need.

In some embodiments, the present disclosure provides a method of increasing the bioavailability of ibogaine in a patient in need thereof, the method comprising administering to the patient: (a) a drug that inhibits the metabolism of ibogaine and (b) a therapeutically effective amount of ibogaine, or a pharmaceutically acceptable salt thereof.

In some embodiments, the present disclosure provides a method of treating a condition that is treatable with ibogaine in a patient in need thereof, the method comprising administering to the patient: (a) a drug that inhibits the metabolism of ibogaine and (b) a therapeutically effective amount of ibogaine, or a pharmaceutically acceptable salt thereof.

In some embodiments, the condition that is treatable with ibogaine is selected from the group consisting of alcoholism, substance abuse disorder, and opioid use disorder. In some embodiments, the condition is opioid use disorder. In some embodiments, the condition that is treatable with ibogaine is symptoms of detoxification and/or withdrawal that result from stopping or reducing the use of a medication or drug. In some embodiments, the medication or drug is a substance with a high potential for dependency or abuse. In some embodiments, the condition that is treatable with ibogaine is a condition related to compulsive/repetitive behaviors, underlying neurocircuitries and neuroplastic effects (e.g., addictions such as gambling or sex, eating disorders, obsessive compulsive disorder (OCD), major depressive disorder (MDD), treatment-resistant depression (TRD), anxiety, post-traumatic stress disorder) (PTSD), attention-deficit/hyperactivity disorder (ADHD), autism spectrum disorder (ASD), and the like).

In some embodiments, the present disclosure provides a method of preventing relapse of a condition that is treatable with ibogaine in a patient in need thereof, the method comprising administering to the patient: (a) a drug that inhibits the metabolism of ibogaine and (b) a therapeutically effective amount of ibogaine, or a pharmaceutically acceptable salt thereof. In some embodiments, the methods of the present disclosure are used to prevent relapse of a substance abuse disorder, including opioid abuse disorder.

In some embodiments of the present method, a daily dose of about 20 mg to about 1000 mg of ibogaine, or a pharmaceutically acceptable salt thereof is administered to the patient, e.g., about 20 mg, about 30 mg, about 40 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg including all ranges and values therebetween. In some embodiments, a daily dose of about 240 mg of ibogaine, or a pharmaceutically acceptable salt thereof is administered to the patient. In some embodiments, a daily dose of about 320 mg of ibogaine, or a pharmaceutically acceptable salt thereof is administered to the patient. In some embodiments, a daily dose of about 400 mg of ibogaine, or a pharmaceutically acceptable salt thereof is administered to the patient.

In some embodiments of the present method, a daily dose of about 10 mg to about 40 mg of ibogaine, or a pharmaceutically acceptable salt thereof is administered to the patient, e.g., about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg, including all ranges and values therebetween. In some embodiments, a daily dose of about 20 mg of ibogaine, or a pharmaceutically acceptable salt thereof is administered to the patient.

In some embodiments of the present method, a daily dose of about 0.1 mg/kg to about 20 mg/kg of ibogaine, or a pharmaceutically acceptable salt thereof is administered to the patient, e.g., about 0.1 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, including all ranges and values therebetween. In some embodiments, a daily dose of about 2 mg/kg to about 12 mg/kg of ibogaine, or a pharmaceutically acceptable salt thereof is administered to the patient. In some embodiments, a daily dose of about 2 mg/kg of ibogaine, or a pharmaceutically acceptable salt thereof is administered to the patient. In some embodiments, a daily dose of about 4 mg/kg of ibogaine, or a pharmaceutically acceptable salt thereof is administered to the patient. In some embodiments, a daily dose of about 6 mg/kg of ibogaine, or a pharmaceutically acceptable salt thereof is administered to the patient. In some embodiments, a daily dose of about 8 mg/kg of ibogaine, or a pharmaceutically acceptable salt thereof is administered to the patient. In some embodiments, a daily dose of about 10 mg/kg of ibogaine, or a pharmaceutically acceptable salt thereof is administered to the patient. In some embodiments, a daily dose of about 12 mg/kg of ibogaine, or a pharmaceutically acceptable salt thereof is administered to the patient.

In some embodiments, the drug that inhibits the metabolism of ibogaine is a CYP2D6 inhibitor. In some embodiments, the CYP2D6 inhibitor is selected from the group consisting of abiraterone, amiodarone, bupropion, celecoxib, chloroquine, chlorpromazine, cimetidine, cinacalcet, citalopram, clobazam, clozapine, cobicistat, desvenlafaxine, diltiazem, diphenhydramine, doxorubicin, duloxetine, Echinacea, escitalopram, febuxostat, fluoxetine, fluphenazine, Gingko biloba, fluvoxamine, gefitinib, haloperidol, hydralazine, hydroxychloroquine, imatinib, labetalol, lansoprazole, lorcaserin, metoclopramide, methadone, mirabegron, olanzapine, Panax ginseng, paroxetine, pazopanib, perhexiline, propafenone, progesterone, propoxyphene, quinidine, ranitidine, risperidone, ritonavir, sertraline, telithromycin, terbinafine, terfenadine, testosterone, thioridazine, trifluperidol, verapamil, vemurafenib. In some embodiments, the CYP2D6 inhibitor is an enantiomer of one of the CYP2D6 inhibitor compounds disclosed herein.

In some embodiments, a dose of about 1 mg/kg to about 20 mg/kg of a CYP2D6 inhibitor is administered to the patient, e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, including all ranges and values therebetween. In some embodiments, a dose of about 1 mg/kg to about 5 mg/kg of a CYP2D6 inhibitor is administered to the patient. In some embodiments, a dose of about 1 mg/kg of a CYP2D6 inhibitor is administered to the patient. In some embodiments, a dose of about 2 mg/kg of a CYP2D6 inhibitor is administered to the patient. In some embodiments, a dose of about 3 mg/kg of a CYP2D6 inhibitor is administered to the patient. In some embodiments, a dose of about 4 mg/kg of a CYP2D6 inhibitor is administered to the patient. In some embodiments, a dose of about 5 mg/kg of a CYP2D6 inhibitor is administered to the patient. In some embodiments, a dose of about 6 mg/kg of a CYP2D6 inhibitor is administered to the patient.

In some embodiments, the CYP2D6 inhibitor is bupropion. Bupropion is a reversible inhibitor of CYP2D6 with a single-dose half-life of 10-15h and metabolites that also inhibit CYP2D6 (hydroxybupropion half-life: 22-25 h; other active metabolites half-lives: 27-60 h) (Sager et al., 2017; Connarn et al., 2017). In vivo CYP2D6 inhibition in humans was demonstrated in the context of chronic bupropion administration (Kotylar et al., 2005). In some embodiments, a dose of about 50 mg to about 250 mg of bupropion is administered to the patient, e.g., about 50 mg, about 75 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg, including all ranges and values therebetween. In some embodiments, a dose of about 100 mg of bupropion is administered to the patient. In some embodiments, a dose of about 150 mg of bupropion is administered to the patient. In some embodiments, a dose of about 200 mg of bupropion is administered to the patient. In some embodiments, the dose of bupropion is administered once daily. In some embodiments, the dose of bupropion is administered twice daily.

In some embodiments, the CYP2D6 inhibitor is fluoxetine or a pharmaceutically acceptable salt thereof. Fluoxetine is a reversible inhibitor of CYP2D6 with a long half-life (days) that has been shown to inhibit CYP2D6 in humans after single administration (Jeppesen et al., 1996). In some embodiments, a dose of about 5 mg to about 150 mg of fluoxetine or a pharmaceutically acceptable salt thereof is administered to the patient, e.g., about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, or about 150 mg, including all ranges and values therebetween. In some embodiments, a dose of about 20 mg of fluoxetine or a pharmaceutically acceptable salt thereof is administered to the patient. In some embodiments, a dose of about 40 mg of fluoxetine or a pharmaceutically acceptable salt thereof is administered to the patient. In some embodiments, a dose of about 60 mg of fluoxetine or a pharmaceutically acceptable salt thereof is administered to the patient. In some embodiments, a dose of about 80 mg of fluoxetine or a pharmaceutically acceptable salt thereof is administered to the patient. In some embodiments, the dose of fluoxetine or a pharmaceutically acceptable salt thereof is administered once daily. In some embodiments, the dose of fluoxetine or a pharmaceutically acceptable salt thereof is administered twice daily.

In some embodiments, the CYP2D6 inhibitor is quinidine or a pharmaceutically acceptable salt thereof. Quinidine, a potent, reversible, competitive CYP2D6 inhibitor, can inhibit the metabolism of the CYP2D6 substrate debrisoquine in vivo to the extent that extensive metabolizer subjects receiving quinidine demonstrate debrisoquine pharmacokinetics phenotypically similar to those exhibited in CYP2D6 poor metabolizers (Brosen et al., 1987). Quinidine has been used in a fixed dose combination with dextromethorphan (Nuedexta®), designed specifically to inhibit CYP2D6-metabolism of dextromethorphan and increase its bioavailability. In some embodiments, a dose of about 1 mg to about 20 mg of quinidine or a pharmaceutically acceptable salt thereof is administered to the patient, e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg, including all ranges and values therebetween. In some embodiments, a dose of about 5 mg of quinidine or a pharmaceutically acceptable salt thereof is administered to the patient. In some embodiments, a dose of about 10 mg of quinidine or a pharmaceutically acceptable salt thereof is administered to the patient. In some embodiments, a dose of about 20 mg of quinidine or a pharmaceutically acceptable salt thereof is administered to the patient. In some embodiments, the dose of quinidine or a pharmaceutically acceptable salt thereof is administered once daily. In some embodiments, the dose of quinidine or a pharmaceutically acceptable salt thereof is administered twice daily.

In some embodiments, the CYP2D6 inhibitor is administered before the first dose of ibogaine or a pharmaceutically acceptable salt thereof. In some embodiments, the CYP2D6 inhibitor is administered as a single dose before the first dose of ibogaine or a pharmaceutically acceptable salt thereof. In some embodiments, the CYP2D6 inhibitor is administered within about 1 h, about 2 h, about 3 h, about 4 h, about 5 h, about 6 h, about 7 h , about 8 h, about 9 h, about 10 h, about 11 h, about 12 h, about 13 h, about 14 h or about 15 h of administration of the ibogaine or a pharmaceutically acceptable salt thereof. In some embodiments, the CYP2D6 inhibitor is administered within about 12 h of administration of the ibogaine or a pharmaceutically acceptable salt thereof. In some embodiments, the CYP2D6 inhibitor is administered 1 to 10 days (e.g., 1 day, 2 days, 3 days, 4 days, 5 days, 6 days, 7 days, 8 days, 9 days, or 10 days) prior to administration of the ibogaine or a pharmaceutically acceptable salt thereof. In some embodiments, the CYP2D6 inhibitor is administered 5 to 7 days prior to administration of the ibogaine or a pharmaceutically acceptable salt thereof. In some embodiments, the CYP2D6 inhibitor is administered as a single daily dose for 1 day, 2 days, 3 days, 4 days, 5 days, 6 days, 7 days, 8 days, 9 days or 10 days before the first dose of ibogaine or a pharmaceutically acceptable salt thereof. In some embodiments, the CYP2D6 inhibitor is co-administered (e.g., concomitantly or concurrently) with the ibogaine or a pharmaceutically acceptable salt thereof. In some embodiments, the CYP2D6 inhibitor is administered before each dose of ibogaine or a pharmaceutically acceptable salt thereof. In some embodiments, the CYP2D6 inhibitor is administered before a dose of ibogaine or a pharmaceutically acceptable salt thereof as needed.

In some embodiments, the drug that inhibits the metabolism of ibogaine is a CYP2D6 inactivator. In some embodiments, a dose of about 1 mg/kg to about 20 mg/kg of a CYP2D6 inactivator is administered to the patient, e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, including all ranges and values therebetween. In some embodiments, a dose of about 1 mg/kg to about 5 mg/kg of a CYP2D6 inactivator is administered to the patient. In some embodiments, a dose of about 1 mg/kg of a CYP2D6 inactivator is administered to the patient. In some embodiments, a dose of about 2 mg/kg of a CYP2D6 inactivator is administered to the patient. In some embodiments, a dose of about 3 mg/kg of a CYP2D6 inactivator is administered to the patient. In some embodiments, a dose of about 4 mg/kg of a CYP2D6 inactivator is administered to the patient. In some embodiments, a dose of about 5 mg/kg of a CYP2D6 inactivator is administered to the patient. In some embodiments, a dose of about 6 mg/kg of a CYP2D6 inactivator is administered to the patient.

In some embodiments, the CYP2D6 inactivator is selected from the group consisting of MDMA, paroxetine, cimetidine, pimozide, methamphetamine, metoclopramide or desethylamiodarone.

In some embodiments, a dose of about 1 mg/kg to about 10 mg/kg of MDMA is administered to the patient, e.g., about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg, including all ranges and values therebetween. In some embodiments, a dose of about 1 mg/kg to about 5 mg/kg of a MDMA is administered to the patient. In some embodiments, a dose of about 1 mg/kg of MDMA is administered to the patient. In some embodiments, a dose of about 2 mg/kg of MDMA is administered to the patient. In some embodiments, a dose of about 3 mg/kg of MDMA is administered to the patient. In some embodiments, a dose of about 4 mg/kg of MDMA is administered to the patient. In some embodiments, a dose of about 5 mg/kg of MDMA is administered to the patient. In some embodiments, a dose of about 6 mg/kg of MDMA is administered to the patient. In some embodiments, the dose of MDMA is administered once daily. In some embodiments, the dose of MDMA is administered once daily for 1 day, 2 days, or 3 days prior to administration of ibogaine or a pharmaceutically acceptable salt thereof.

In some embodiments, a dose of about 5 mg to about 150 mg of paroxetine is administered to the patient, e.g., about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, or about 150 mg, including all ranges and values therebetween. In some embodiments, a dose of about 10 mg of paroxetine is administered to the patient. In some embodiments, a dose of about 40 mg of paroxetine is administered to the patient. In some embodiments, a dose of about 20 mg of paroxetine is administered to the patient. In some embodiments, a dose of about 40 mg of paroxetine is administered to the patient. In some embodiments, a dose of about 60 mg of paroxetine is administered to the patient. In some embodiments, a dose of about 80 mg of paroxetine is administered to the patient. In some embodiments, the dose of paroxetine is administered once daily. In some embodiments, the dose of paroxetine is administered once daily for 1 day, 2 days, 3 days, 4 days, 5 days, or 6 days prior to administration of ibogaine or a pharmaceutically acceptable salt thereof.

In some embodiments, the CYP2D6 inactivator is co-administered (e.g., concomitantly or concurrently) with the ibogaine or a pharmaceutically acceptable salt thereof. In some embodiments, the CYP2D6 inactivator is administered at least 12 h, at least 1 day, at least 2 days, at least 3 days, or at least 4 days prior to the administration of ibogaine or a pharmaceutically acceptable salt thereof. In some embodiments, the CYP2D6 inactivator is administered at least 1 day prior to the administration of ibogaine or a pharmaceutically acceptable salt thereof. In some embodiments, the CYP2D6 inactivator is administered about 1 day prior to the administration of ibogaine or a pharmaceutically acceptable salt thereof. In some embodiments, the CYP2D6 inactivator is administered about 12 h to about 18 h prior to the administration of ibogaine or a pharmaceutically acceptable salt thereof. In some embodiments, the CYP2D6 inactivator is administered as a one-time single dose prior to administration of a first dose of ibogaine or a pharmaceutically acceptable salt thereof. In some embodiments, the administration of ibogaine or a pharmaceutically acceptable salt thereof is of a first dose ibogaine or a pharmaceutically acceptable salt thereof. In some embodiments, the CYP2D6 inactivator is administered as a single dose prior to administration of a dose of ibogaine or a pharmaceutically acceptable salt thereof as needed. In some embodiments, the CYP2D6 inactivator is administered as a one-time single dose prior to administration of a dose of ibogaine or a pharmaceutically acceptable salt thereof as needed.

In some embodiments, the patient is pre-treated with a drug that inhibits ibogaine metabolism prior to administration of the ibogaine. In some embodiments, the patient is pre-treated with a drug that inhibits ibogaine metabolism at least 1 day, at least 2 days, at least 3 days, at least 4 days, or at least five days prior to administration of ibogaine or a pharmaceutically acceptable salt thereof. In some embodiments, the patient is pre-treated with a drug that inhibits ibogaine metabolism at least 3 days prior (e.g., 5 days prior or 7 days prior) to administration of ibogaine or a pharmaceutically acceptable salt thereof.

In some embodiments, a therapeutically effective dose of ibogaine is administered in combination with a drug that inhibits the metabolism (i.e., a CYP2D6 inhibitor or a CYP2D6 inactivator) of ibogaine. In some embodiments, the therapeutically effective dose of ibogaine administered in combination with a drug that inhibits the metabolism is lower than the same dose of ibogaine without administration of the drug that inhibits the metabolism of ibogaine. In some embodiments, the therapeutically effective dose is about 5%, about 6%, about 7%, about 8%, about 9%, about 10%, about 12%, about 14%, about 16%, about 18%, about 20%, about 22%, about 24%, about 26%, about 28%, about 30%, about 32%, about 34%, about 36%, about 38%, about 40%, about 42%, about 44%, about 46%, about 48%, or about 50%, including all ranges and values therebetween.

In some embodiments, the administration of the drug that inhibits the metabolism of ibogaine reduces the patient's systemic exposure to noribogaine compared to a patient administered the same dose of ibogaine without administration of the drug that inhibits the metabolism of ibogaine. In some embodiments, the patient's systemic exposure to noribogaine is reduced by about 5%, about 6%, about 7%, about 8%, about 9%, about 10%, about 12%, about 14%, about 16%, about 18%, about 20%, about 22%, about 24%, about 26%, about 28%, about 30%, about 32%, about 34%, about 36%, about 38%, about 40%, about 42%, about 44%, about 46%, about 48%, or about 50%, including all ranges and values therebetween.

In some embodiments, the administration of the drug that inhibits the metabolism of ibogaine increases the patient's systemic exposure to ibogaine compared to a patient administered the same dose of ibogaine without administration of the drug that inhibits the metabolism of ibogaine. In some embodiments, the patient's systemic exposure to ibogaine is increased by about 5%, about 6%, about 7%, about 8%, about 9%, about 10%, about 12%, about 14%, about 16%, about 18%, about 20%, about 22%, about 24%, about 26%, about 28%, about 30%, about 32%, about 34%, about 36%, about 38%, about 40%, about 42%, about 44%, about 46%, about 48%, or about 50%, including all ranges and values therebetween.

In some embodiments, the administration of the drug that inhibits the metabolism of ibogaine reduces the patient's Cmax of noribogaine compared to a patient administered the same dose of ibogaine without administration of the drug that inhibits the metabolism of ibogaine. In some embodiments, the patient's Cmax of noribogaine is decreased by about 5%, about 6%, about 7%, about 8%, about 9%, about 10%, about 12%, about 14%, about 16%, about 18%, about 20%, about 22%, about 24%, about 26%, about 28%, or about 30%, including all ranges and values therebetween.

In some embodiments, the administration of the drug that inhibits the metabolism of ibogaine increases the patient's Cmax of ibogaine compared to a patient administered the same dose of ibogaine without administration of the drug that inhibits the metabolism of ibogaine. In some embodiments, the patient's Cmax of ibogaine is increased by about 5%, about 6%, about 7%, about 8%, about 9%, about 10%, about 12%, about 14%, about 16%, about 18%, about 20%, about 22%, about 24%, about 26%, about 28%, or about 30%, including all ranges and values therebetween.

### NUMBERED EMBODIMENTS OF THE DISCLOSURE

In addition to the disclosure above, the Examples below, and the appended claims, the disclosure sets forth the following numbered embodiments.
1. A method of increasing and prolonging exposure to ibogaine in a patient, while reducing exposure to noribogaine and associated risk of QT prolongation comprising administering to the patient:
   (a) a drug that inhibits the metabolism of ibogaine and
   (b) an effective amount of ibogaine, or a pharmaceutically acceptable salt thereof.
2. A method of increasing the bioavailability of ibogaine in a patient in need thereof, the method comprising administering to the patient:
   (a) a drug that inhibits the metabolism of ibogaine; and
   (b) an effective amount of ibogaine, or a pharmaceutically acceptable salt thereof.
3. A method of treating a condition that is treatable with ibogaine in a patient in need thereof, the method comprising administering to the patient:
   (a) a drug that inhibits the metabolism of ibogaine; and
   (b) a therapeutically effective amount of ibogaine, or a pharmaceutically acceptable salt thereof.
4. The method of embodiment 3, wherein the condition is alcoholism, substance abuse disorder, or opioid use disorder.
5. The method of embodiment 4, wherein the condition is opioid use disorder.
6. The method of any one of embodiments 1-5, wherein a daily dose of about 20 mg of ibogaine, or a pharmaceutically acceptable salt thereof is administered to the patient.
7. The method of any one of embodiments 1-6, wherein the drug that inhibits the metabolism of ibogaine is a CYP2D6 inhibitor.
8. The method of embodiment 7, wherein the CYP2D6 inhibitor is abiraterone, amiodarone, bupropion, celecoxib, chloroquine, chlorpromazine, cimetidine, cinacalcet, citalopram, clobazam, clozapine, cobicistat, desvenlafaxine, diltiazem, diphenhydramine, doxorubicin, duloxetine, Echinacea, escitalopram, febuxostat, fluoxetine, fluphenazine, Gingko biloba, fluvoxamine, gefitinib, haloperidol, hydralazine, hydroxychloroquine, imatinib, labetalol, lansoprazole, lorcaserin, metoclopramide, methadone, mirabegron, olanzapine, Panax ginseng, paroxetine, pazopanib, perhexiline, propafenone, progesterone, propoxyphene, quinidine, ranitidine, risperidone, ritonavir, sertraline, telithromycin, terbinafine, terfenadine, testosterone, thioridazine, trifluperidol, verapamil, or vemurafenib.
9. The method of embodiment 7, wherein the CYP2D6 inhibitor is bupropion.
10. The method of embodiment 7, wherein the CYP2D6 inhibitor is fluoxetine.
11. The method of embodiment 7, wherein the CYP2D6 inhibitor is quinidine.
12. The method of any one of embodiments 6-11, comprising administering the CYP2D6 inhibitor within about 12 h of administration of ibogaine or a pharmaceutically acceptable salt thereof.
13. The method of any one of embodiments 6-11, comprising co-administering the CYP2D6 inhibitor with the ibogaine or a pharmaceutically acceptable salt thereof.
14. The method of any one of embodiments 1-6, wherein the drug that inhibits the metabolism of ibogaine is a CYP2D6 inactivator.
15. The method of embodiment 14, wherein the CYP2D6 inactivator is MDMA, paroxetine, cimetidine, pimozide, methamphetamine, metoclopramide or desethylamiodarone.
16. The method of embodiment 14 or 15, comprising administering the CYP2D6 inactivator at least 1 day prior to ibogaine or a pharmaceutically acceptable salt thereof.
17. The method of embodiment 14 or 15, comprising co-administering the CYP2D6 inactivator with the ibogaine or a pharmaceutically acceptable salt thereof.
18. The method of any one of embodiments 1-17, comprising pre-treating the patient with the drug that inhibits ibogaine metabolism prior to administration of the ibogaine or a pharmaceutically acceptable salt thereof.
19. The method of embodiment 18, comprising pre-treating the patient with the drug that inhibits ibogaine metabolism for at least 3 days prior to administration of the ibogaine or a pharmaceutically acceptable salt thereof.
20. The method of embodiment 18, comprising pre-treating the patient with the drug that inhibits ibogaine metabolism for about 5 days prior to administration of the ibogaine or a pharmaceutically acceptable salt thereof.
21. The method of any one of embodiments 1-20, wherein the administration of the drug that inhibits the metabolism of ibogaine reduces the patient's systemic exposure to noribogaine compared to a patient administered the same dose of ibogaine without administration of the drug that inhibits the metabolism of ibogaine.
22. The method of any one of embodiments 1-20, wherein the administration of the drug that inhibits the metabolism of ibogaine reduces the patient's Cmax exposure to noribogaine compared to a patient administered the same dose of ibogaine without administration of the drug that inhibits the metabolism of ibogaine.

### EXAMPLES

Example 1. Pharmacokinetic Studies with CYP2D6 Inhibitors

Pharmacokinetic (PK) studies in rats, dogs and/or nonhuman primates:

Part 1a

Objective: To determine extent that reduced CYP2D6 activity increases systemic exposure to ibogaine and reduces systemic exposure to noribogaine in animals.

Design: Evaluate plasma (and brain, in rat only) ibogaine and noribogaine concentrations using LC-MS/MS methods, and determine PK parameters (Cmax, Tmax, area under the curve (AUC)) in animals treated for 5 consecutive days with a single dose of vehicle or CYP2D6 inhibitor (e.g., bupropion, fluoxetine, or quinidine) per day (to achieve steady-state concentrations) followed on day 5 by a single dose of ibogaine known to exhibit safety and/or therapeutic-like effects in the species being evaluated. Multiple dose levels of CYP2D6 inhibitor may be tested to characterize dose-response functions.

Analysis: PK parameters under investigation include maximum concentration (Cmax), time to maximum concentration (Tmax) and area under the curve (AUC) for ibogaine and noribogaine.

Part 1b

Objective/Design: Additional studies include the testing of coadministration of single or multiple once-daily doses of a CYP2D6 inhibitor or vehicle in combination with a dose of ibogaine known to exhibit safety and/or therapeutic-like effects in a particular species, in order to determine PK exposure parameters associated with single or multiple once-daily coadministrations. Multiple dose levels of CYP2D6 inhibitor may be tested to characterize dose-response functions.

### Example 2. Pharmacokinetic Studies with CYP2D6 Inactivators

Part 2a

Objective: To confirm that a lack of CYP2D6 activity substantially increases systemic exposure to ibogaine while decreasing systemic exposure to noribogaine in animals.

Design: Evaluate plasma (and brain, in rat only) ibogaine and noribogaine concentrations and determine PK parameters in animals treated with a single dose of vehicle or CYP2D6 inactivator (e.g., MDMA, paroxetine, cimetidine, pimozide, methamphetamine, metoclopramide or desethylamiodarone) followed 24 hours later by a single dose of ibogaine known to exhibit safety and/or therapeutic-like effects in that species. Multiple dose levels of CYP2D6 inactivator may be tested to characterize dose-response functions.

### Part 2b

Objective/Design: Additional studies may include co-administering a single dose of CYP2D6 inactivator (or vehicle) in combination with a single dose of ibogaine known to exhibit safety and/or therapeutic-like effects in a particular species, in order to determine ibogaine and noribogaine PK exposure parameters (Cmax, Tmax, and AUC) associated with a single coadministration. Multiple dose levels of CYP2D6 inactivator may be tested to characterize dose-response functions.

Bioanalytical methods for identification and quantification of ibogaine and noribogaine in biological fluids are known in the art and will be utilized in these studies (see Hearn, W. L., Pablo, J., Hime, G. W., and Mash, D. C. (1995) Identification and quantification of ibogaine and an o-demethylated metabolite in brain and biological fluids using gas chromatography-mass spectrometry. J. Anal. Toxicol. 19, 427-434, which is incorporated herein by reference in its entirety).

Nonclinical proof-of-concept pharmacokinetic (PK) studies (rats, dogs and/or nonhuman primates):

To determine if a lack of CYP2D6 activity substantially increases ibogaine exposures and PK parameters in animals, plasma (and brain, in rat only) ibogaine and noribogaine exposures and PK parameters in animals treated with a single dose of vehicle or CYP2D6 inhibitor (inhibitor including, but not limited to inactivator) followed by a single dose of ibogaine are evaluated. Studies include the testing of multiple single doses of CYP2D6 inhibitor followed by administration of a dose of ibogaine known to exhibit safety and/or therapeutic-like effects in a particular species, in order to characterize the CYP2D6 inhibitor dose-response function.

Nonclinical proof-of-concept PD studies (rats and/or nonhuman primates):

To determine if a lack of CYP2D6 activity substantially increases ibogaine's therapeutic-like effects in animals, drug self-administration behavior (e.g., opioids, cocaine, ethanol) in animals pretreated with a single dose of vehicle or CYP2D6 inhibitor followed by a single dose of ibogaine is evaluated. Dose(s) of CYP2D6 inhibitor based on prior PK studies in the same species are tested. Intravenous drug self-administration studies involve training the animal to emit a response (e.g., lever press) for a non-drug reinforcer (e.g., food) under specific stimulus conditions in the test chamber. Then, the animal undergoes aseptic surgery to place a chronically indwelling intravenous cannula that is connected to a drug infusion system within the test chamber. After surgical recovery, the stimulus conditions in the test chamber now signal the availability of a drug reinforcer (e.g., morphine or cocaine), and lever pressing results in an intravenous infusion of the available drug during daily session. For studies of oral self-administration (e.g., ethanol), animals are trained to self-administer the drug using a lever paradigm (e.g., in which pressing of the lever results in delivery of the drug solution into a drinking cup) or a two-bottle choice IDF v1.0 paradigm (e.g., in which animals are allowed to drink for 24 hours from one bottle containing a drug solution or another bottle containing water). During a test session, vehicle or a dose of CYP2D6 inhibitor is administered prior to a dose of ibogaine and then, responses on the drug-associated lever or the volume of drug solution consumed are measured. CYP2D6 inhibition increases ibogaine's therapeutic-like effects (i.e., to reduce drug self-administration) and demonstrates that a given administered dose of ibogaine enhances efficacy in the presence of the CYP2D6 inhibitor.

Clinical proof-of-concept PK study:

To determine if a lack of CYP2D6 activity substantially increases ibogaine exposures and PK parameters in humans, plasma ibogaine and noribogaine exposures and PK parameters in subjects treated with a single dose of placebo or CYP2D6 inhibitor followed by a single dose of ibogaine are evaluated.

### REFERENCES

Belgers, M., Leenaars, M., Homberg, J. R., Ritskes-Hoitinga, M., Schellekens, A. F. A., and Hooijmans, C. R. (2016). Ibogaine and addiction in the animal model, a systematic review and meta-analysis. Transl. Psychiatry 6, e826. doi:10.1038/tp.2016.71

Brosen, K., Gram, L. F., Haghfelt, T., and Bertilsson, L. (1987). Extensive metabolizers of debrisoquine become poor metabolizers during quinidine treatment. Pharmacol. Toxicol. 60(4), 312-314.

Cascorbi I. (2003) Pharmacogenetics of cytochrome p4502D6: genetic background and clinical implication. Eur J Clin Invest 33 (Suppl 2):17-22.

Connarn, J. N., Flowers, S., Kelly, M., Luo, R., Ward, K. M., Harrington, G., Moncion, I., Kamali, M., McInnis, M., Feng, M. R., Ellingrod, V., Babiskin, A., Zhang, X., and Sun, D. (2017). Pharmacokinetics and pharmacogenomics of bupropion in three different formulations with different release kinetics in healthy human volunteers. AAPS J. 19(5), 1513-1522.

De Gregori M., Allegri M., De Gregori S., Garbin G., Tinelli C., Regazzi M., Govoni S., and Ranzani G. N. (2010) How and why to screen for CYP2D6 interindividual variability in patients under pharmacological treatments. Curr. Drug Metab. 11:276-282.

De la Torre, R., Yubero-Lahoz, S., Pardo-Lozano, R., and Farre, M. (2012). MDMA, methamphetamine, and CYP2D6 pharmacogenetics: what is clinically relevant? Front. Genetics 3, 235. doi: 10.3389/fgene.2012.00235

Glue, P., Winter, H., Garbe, K., Jakobi, H., Lyudin, A., Lenagh-Glue, Z. and Hung, C. T. (2015). Influence of CYP2D6 activity on the pharmacokinetics and pharmacodynamics of a single 20 mg dose of ibogaine in healthy volunteers. J. Clin. Pharmacol. 55(6), 680-687.

Gopisankar, M. G. (2017). CYP2D6 pharmacogenomics. Egyptian J. Med. Human Genetics. 18, 309-313.

Hearn, W. L., Pablo, J., Hime, G. W., and Mash, D. C. (1995) Identification and quantification of ibogaine and an o-demethylated metabolite in brain and biological fluids using gas chromatography-mass spectrometry. J. Anal. Toxicol. 19, 427-434.

Henstra, M., Wong, L., Chahbouni, A., Swart, N., Allaart, C., and Sombogaard, F. (2017). Toxicokinetics of ibogaine and noribogaine in a patient with prolonged multiple cardiac arrhythmias after ingestion of internet purchased ibogaine. Clin. Toxicol. 55(6), 600-602.

Jeppesen, U., Gram, L. F., Vistisen, K., Loft, S., Poulsen, H. E., and Brosen, K. (1996). Dose-dependent inhibition of CYP1A2, CYP2C19 and CYP2D6 by citalopram, fluoxetine, fluvoxamine and paroxetine. Eur. J. Clin. Pharmacol. 51, 73-78.

Koenig, X., and Hilber, K. (2015). The anti-addiction drug ibogaine and the heart: a delicate relation. Molecules 20, 2208-2228.

Kotlyar, M., Brauer, L. H., Tracy, T. S., Hatsukami, D. K., Harris, J., Bronars, C. A., and Adson, D. E. (2005). Inhibition of CYP2D6 activity by bupropion. J. Clin. Psychopharmacol. 25(3), 226-229.

Mash, D. C., Duque, L., Page, B., and Allen-Ferdinand, K. (2018). Ibogaine detoxification transitions opioid and cocaine abusers between dependence and abstinence: clinical observations and treatment outcomes. Front. Pharmacol. 9, 529. doi: 10.3389/fphar.2018.00529

Mash, D. C., Kovera, C. A., Pablo, J., Tyndale, R., Ervin, F. R., Kamlet, J. D., and Hearn, W. L. (2001). Ibogaine in the treatment of heroin withdrawal. Alkaloids Chem. Biol. 56, 155-171.

Obach, R. S., Pablo, J., and Mash, D. C. (1998). Cytochrome P4502D6 catalyzes the o-demethylation of the psychoactive alkaloid ibogaine to 12-hydroxyibogamine. Drug Metab. Disposition 25, 1359-1369.

Sager, J. E., Tripathy, S., Price, L. S. L., Nath, A., Chang, J., Stephenson-Famy, A., and Isoherranen, N. (2017). In vitro to in vivo extrapolation of the complex drug-drug interaction of bupropion and its metabolites with CYP2D6; simultaneous reversible inhibition and CYP2D6 downregulation. Biochem. Pharmacol. 123, 85-96.

Wasko, M. J., Witt-Enderby, P. A., and Surratt, C. K. (2018). DARK classics in chemical neuroscience: ibogaine. ACS Chem. Neurosci. 9, 2475-2483.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

1. A method of increasing and prolonging exposure to ibogaine in a patient, while reducing exposure to noribogaine and associated risk of QT prolongation comprising administering to the patient:
(a) a drug that inhibits the metabolism of ibogaine; and
(b) an effective amount of ibogaine, or a pharmaceutically acceptable salt thereof.

2. A method of treating a condition that is treatable with ibogaine in a patient in need thereof, the method comprising administering to the patient:
(a) a drug that inhibits the metabolism of ibogaine; and
(b) a therapeutically effective amount of ibogaine, or a pharmaceutically acceptable salt thereof.

3. The method of claim 2, wherein the condition is alcoholism, substance abuse disorder, or opioid use disorder.

4. The method of claim 3, wherein the condition is opioid use disorder.

5. The method of claim 4, wherein a daily dose of about 20 mg of ibogaine, or a pharmaceutically acceptable salt thereof is administered to the patient.

6. The method of claim 1, wherein the drug that inhibits the metabolism of ibogaine is a CYP2D6 inhibitor.

7. The method of claim 6, wherein the CYP2D6 inhibitor is abiraterone, amiodarone, bupropion, celecoxib, chloroquine, chlorpromazine, cimetidine, cinacalcet, citalopram, clobazam, clozapine, cobicistat, desvenlafaxine, diltiazem, diphenhydramine, doxorubicin, duloxetine, Echinacea, escitalopram, febuxostat, fluoxetine, fluphenazine, Gingko biloba, fluvoxamine, gefitinib, haloperidol, hydralazine, hydroxychloroquine, imatinib, labetalol, lansoprazole, lorcaserin, metoclopramide, methadone, mirabegron, olanzapine, Panax ginseng, paroxetine, pazopanib, perhexiline, propafenone, progesterone, propoxyphene, quinidine, ranitidine, risperidone, ritonavir, sertraline, telithromycin, terbinafine, terfenadine, testosterone, thioridazine, trifluperidol, verapamil, or vemurafenib.

8. The method of claim 6, wherein the CYP2D6 inhibitor is bupropion.

9. The method of claim 6, wherein the CYP2D6 inhibitor is fluoxetine.

10. The method of claim 6, wherein the CYP2D6 inhibitor is quinidine.

11. The method of claim 6, comprising administering the CYP2D6 inhibitor within about 12 hours of administration of ibogaine or a pharmaceutically acceptable salt thereof.

12. The method of claim 6, comprising administering the CYP2D6 inhibitor with the ibogaine or a pharmaceutically acceptable salt thereof.

13. The method of claim 1, wherein the drug that inhibits the metabolism of ibogaine is a CYP2D6 inactivator.

14. The method of claim 13, wherein the CYP2D6 inactivator is 3,4-methylenedioxymethamphetamine (MDMA), paroxetine, cimetidine, pimozide, methamphetamine, metoclopramide or desethylamiodarone.

15. The method of claim 13, comprising administering the CYP2D6 inactivator at least 1 day prior to administration of the ibogaine or a pharmaceutically acceptable salt thereof.

16. The method of claim 13, comprising co-administering the CYP2D6 inactivator with the ibogaine or a pharmaceutically acceptable salt thereof.

17. The method of claim 1, comprising pre-treating the patient with the drug that inhibits ibogaine metabolism prior to administration of the ibogaine or a pharmaceutically acceptable salt thereof.

18. The method of claim 17, comprising pre-treating the patient with the drug that inhibits ibogaine metabolism for at least 3 days prior to administration of the ibogaine or a pharmaceutically acceptable salt thereof.

19. The method of claim 17, comprising pre-treating the patient with the drug that inhibits ibogaine metabolism for about 5 days prior to administration of the ibogaine or a pharmaceutically acceptable salt thereof.

20. The method of claim 1, wherein the administration of the drug that inhibits the metabolism of ibogaine reduces the patient's systemic exposure to noribogaine compared to a patient administered an effective amount of ibogaine without administration of the drug that inhibits the metabolism of ibogaine.

21. The method of claim 1, wherein the administration of the drug that inhibits the metabolism of ibogaine reduces the patient's noribogaine Cₘₐₓ compared to a patient administered an effective amount of ibogaine without administration of the drug that inhibits the metabolism of ibogaine.

22. A method of increasing the bioavailability of ibogaine in a patient in need thereof comprising administering to the patient:
(a) a drug that inhibits the metabolism of ibogaine; and
(b) an effective amount of ibogaine, or a pharmaceutically acceptable salt thereof.

23. The method of claim 21, wherein the patient's noribogaine Cₘₐₓ is reduced by about 5% to about 30% compared to the patient administered the effective amount of ibogaine without administration of the drug that inhibits the metabolism of ibogaine.

24. The method of claim 1, wherein the administration of the drug that inhibits the metabolism of ibogaine increases the patient's ibogaine Cₘₐₓ compared to a patient administered an effective amount of ibogaine without administration of the drug that inhibits the metabolism of ibogaine.

25. The method of claim 24, wherein the patient's ibogaine Cₘₐₓ is increased by about 5% to about 30% compared to a patient administered the effective amount of ibogaine without administration of the drug that inhibits the metabolism of ibogaine.

26. The method of claim 1, wherein the effective amount of ibogaine administered in combination with a drug that inhibits the metabolism is lower than an effective amount of ibogaine without administration of the drug that inhibits the metabolism of ibogaine.

27. The method of claim 26, wherein the effective amount of ibogaine is about 5% to about 50% lower than an effective amount of ibogaine without administration of the drug that inhibits the metabolism of ibogaine.

28. The method of claim 1, wherein the effective amount of ibogaine is about 20 mg to about 1000 mg.

29. The method of claim 1, wherein the effective amount of ibogaine is about 10 mg to about 40 mg.
